(19) United States
(12) Patent Application Publication
(10) Pub. No.: US 2003/0082231 A1
(43) Pub. Date: May 1, 2003

(54) Coating for orally administered compositions

(76) Inventors: Cheryl Kos, Gig Harbor, WA (US); Kim Carleton Krumhar, Carlsbad, CA (US); David A. Boyd, Gig Harbor, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/062,749

(22) Filed: Feb. 1, 2002

Related U.S. Application Data
(60) Provisional application No. 60/266,269, filed on Feb. 2, 2001.

(51) Int. Cl. A61K 31/555; A61K 9/36

(52) U.S. Cl. 424/480; 514/185

(57) ABSTRACT

A method for coating a substance with a coating comprising a mixture of a cellulose ether and a metal chlorophyllin chelate is disclosed. The coating can be used for improving stability of the substance and reducing objectionable tastes and/or odors in the substance.

### CROSS REFERENCE TO RELATED APPLICATIONS

This application claims the benefit under 35 U.S.C. §119(e) of U.S. Provisional Application No. 60/266,269, filed Feb. 2, 2001, which is hereby incorporated herein by reference.

### BACKGROUND OF THE INVENTION

1. Field of the Invention

This invention relates to orally administered compositions. More particularly, this invention relates to coatings for orally administered compositions for enhancing stability, taste, and odor of such compositions.

2. Description of the Related Art

Acceptability of many orally administered compositions is affected by stability, taste, and/or odor of the compositions. For example, it is desirable to sequester chemically reactive compounds for preventing unwanted reactions with other compounds. Additionally, formulations that contain unpleasant tastes and/or odors are not readily accepted by consumers. One example of such compounds are sulfur-containing compounds.

In the fields of dietary supplements and nutraceuticals there are numerous compounds and substances that would be more acceptable to consumers if the problems associated with unpleasant tastes and odors could be surmounted. Similarly, there are numerous compounds that could be used more effectively if their tendency to react with other compounds could be controlled.

In pharmaceutical sciences, these problems have been dealt with traditionally by the use of pharmaceutical necessities, which are substances that are of little or no therapeutic value, but are useful in the manufacture and compounding of pharmaceutical preparations. These pharmaceutical necessities include, but are not limited to, antioxidants and preservatives, flavoring agents, and the like.

With reference to flavoring agents, it is well known that there is a close relationship between chemical structure and taste. Solubility, degree of ionization, and type of ions produced in saliva can influence the sensation interpreted by the brain. Sour taste can be caused by hydrogen ions and is proportional to the hydrogen ion concentration and the lipid solubility of the compound. For instance, sour taste is characteristic of acids, tannins, alum, phenols, and lactones. Saltiness can be due to the simultaneous presence of anions and cations, such as, but not limited to, KBr, NH₄Cl, sodium salicylate and the like. High-molecular-weight salts can cause a bitter taste. Free bases, such as alkaloids and amines, such as amphetamines can also give bitter tastes. Polyhydroxy compounds with a molecular weight greater than about 300, halogenated substances, and aliphatic thio compounds can also have bitter tastes. Unsaturation frequently bestows a sharp, biting odor and taste upon compounds. Sweet taste can be due to polyhydroxy compounds, polyhalogenated aliphatic compounds, α-amino acids, and the like. Amino and amide groups, especially if the positive effect is balanced by proximity to a negative group, can also produce a sweet taste. Sweetness increases with the presence of increasing number of hydroxy groups, possibly due to increase in solubility. Amides can be intensely sweet.

No precise relationship between chemical structure and odor has been found. There are no primary odors; also, odors blend into each other. Polymerization can reduce or destroy odor; high valency can give odor and unsaturation can enhance odor. A tertiary carbon atom often can give a camphoraceous odor; esters and lactones can have a fruity odor, and ketones can have a pleasant odor. Strong odors are often accompanied by volatility and chemical reactivity.

A flavoring problem is unique and requires an individual solution. The problem of flavoring is further complicated because flavor and taste depend on individual preferences. Nevertheless, in solving flavoring problems, certain techniques can be used. (1) Blending: Certain flavors blend well with certain tastes to be masked. For example, fruit flavors blend with a sour taste. (2) Overshadow: A flavor with an intensity that is longer and stronger than the obvious taste can be used to overshadow an objectionable taste. (3) Physical: Formation of insoluble compounds of the offending substance, such as coating of tablets, may reduce the flavoring problems. (4) Chemical: Adsorption of the substance on a substrate or forming a complex of the substance with complexing agents may solve the problem. (5) Physiological: The taste buds may be anesthetized by certain flavors such as menthol or mint flavors.

### SUMMARY OF THE INVENTION

Since dietary supplements and nutraceuticals frequently encounter the problems of stability, flavor, and odor, it is not economically feasible to solve each such problem with a unique solution, it would be advantageous to provide a coating for general use with many such substances and that would not offend the sensibilities of consumers of these products who prefer to avoid “unnatural” products.

In view of the foregoing, it will be appreciated that providing a “natural” coating that can be used with dietary supplements and nutraceuticals for solving problems associated with stability, taste, and odor would be a significant advancement in the art. Preferred embodiments are to provide compositions and methods for solving the problems of instability and objectionable flavors and odors often encountered in dietary supplements and nutraceuticals.

A certain embodiment involves a coated substance comprising the substance and a coating surrounding the substance, wherein the coating comprises a mixture of cellulose ether and a metal chlorophyllin chelate.

Another embodiment comprises a method for coating a substance comprising: forming a solution of a cellulose ether; adding a metal chlorophyllin chelate to the cellulose ether solution, thereby forming an chlorophyllin compound/cellulose ether solution; applying the metal chlorophyllin chelate/cellulose ether solution to the substance; and vaporizing the solvent from the metal chlorophyllin chelate/cellulose ether solution, thereby forming a coating on the substance.

### DETAILED DESCRIPTION OF THE EMBODIMENTS

The publications and other reference materials referred to herein to describe the background of the embodiments of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to “a coating” includes reference to two or more of such coatings, reference to “a cellulose ether” comprises reference to one or more of such cellulose ethers, and reference to “an alcohol” includes reference to two or more of such alcohols.

In describing and claiming the certain embodiments of the invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

A coating of the preferred embodiments may be used for a variety of substances. The coating can be used for maintaining stability of a substance and reducing objectionable tastes and/or odors. Examples of substances include, but are not limited to, pharmaceutical compounds, dietary supplements, and nutraceuticals.

A natural coating of the preferred embodiments for use in coating compounds and substances comprises a saccharide polymer, such as, but not limited to, cellulose, starches, and gums. Preferred starches include, but are not limited to, crosslinked starches and modified starches. Preferred gums include, but are not limited to, guar gum and alginates. Preferred cellulose include, but not limited to, methylcellulose ether, hydroxypropyl methylcellulose ether, carboxymethylcellulose ether, sodium carboxymethylcellulose ether, microcrystalline cellulose, and cellobiose.

In certain embodiments, a cellulose ether is used the coating. Preferably, cellulose ether products of the preferred embodiments can be obtained as two basic types: methylcellulose ether and hydroxypropyl methylcellulose ether. Both types of cellulose ethers have the polymeric backbone of cellulose, a natural carbohydrate that contains a basic repeating structure of anhydroglucose units. During the manufacture of cellulose ethers, cellulose fibers are heated with a caustic solution that in turn is treated with methyl chloride, yielding the methyl ether of cellulose. The fibrous reaction product is purified and ground to a fine, uniform powder. Methylcellulose is made using substantially methyl chloride. Examples of methylcellulose are METHOCEL™ A brand products (Dow Chemicals). For hydroxypropyl methylcellulose products, propylene oxide is used in addition to methyl chloride to obtain hydroxypropyl and methyl substitutions on the anhydroglucose units. Examples of hydroxypropyl methylcellulose are METHOCEL™ E, F, J, and K brand products (Dow Chemicals). This substituent group, —OCH₂CH(OH)—CH₃ contains a secondary hydroxyl on a carbon and can also form a propylene glycol ether of cellulose. These products possess varying ratios of hydroxypropyl and methyl substitution, a factor which influences organic solubility and the thermal gelation temperature of aqueous solutions.

The amount of substituent groups on the anhydroglucose units of cellulose can be designated by weight percent or by the average number of substituent groups attached to the ring, a concept known to cellulose chemists as “degree of substitution” (D.S). If all three available positions on each unit are substituted, the D.S. is designated as 3; if an average of two on each ring are reacted, the D.S. is designated as 2, etc. The number of substituent groups on the ring determines the properties of the various products. METHOCEL™ A cellulose ether contains about 27.5 to 31.5% methoxyl, or a methoxyl D.S. of 1.64 to 1.92. In the METHOCEL™ E, METHOCEL™ F, and METHOCEL™ K cellulose ether products, the methoxyl substitution is still the major constituent (see the table below). The molar substitution (MS) reports the number of moles of hydroxypropyl groups per mole of anhydroglucose. In the METHOCEL™ J and 310-Series products, the hydroxypropyl substitution is about 50% of the total substitution.

TABLE 1
Degree of Substitution for METHOCEL ™ Products

| | | | Hydroxy- | |
|---|---|---|---|---|
| | Methoxyl | | propyl | |
| | degree of | | molar sub- | Hydroxy- |
| Product | substitution | Methoxyl % | stitution | propyl |
| METHOCEL ™ A | 1.8 | 30 | — | — |
| METHOCEL ™ E | 1.9 | 28 | 0.23 | 8.5 |
| METHOCEL ™ F | 1.8 | 28 | 0.13 | 5.0 |
| METHOCEL ™ J | 1.3 | 18 | 0.82 | 27 |
| METHOCEL ™ K | 1.4 | 22 | 0.21 | 8.1 |
| METHOCEL ™ | 2.0 | 25 | 0.8 | 25 |
| 310 Series | | | | |

Cellulose ethers can be used in tablet coatings. Cellulose ethers can form strong films with good adhesion. They can provide taste-masking qualities and can act as barriers for water-sensitive drugs or components, while adding no calories. Cellulose ethers also increase compressive strength and reduce friability, yet they increase overall tablet size by a small amount, preferably about 1-3 mm or less. Cellulose ethers have no ionic charge, are stable over a pH range of about 3 to 11, and are enzyme resistant. Also, cellulose ethers can pass through the intestinal tract essentially unchanged, thereby affirming the stability of these compounds to a wide range of biochemical and enzymatic systems. Coatings containing cellulose ethers can be applied in one pan, shorten coating time, reduce skilled operator requirements, and permit the use of automated coating systems.

Cellulose ethers can also be used in the granulation process of making tablets. Used at low concentration as binders in the granulation process, cellulose ethers can produce hard tablets with low friability, while not negatively affecting tablet disintegration. Because cellulose ethers can be used in a wide variety of solvent systems, they are extremely versatile in wet granulation formulations.

In hydrophilic matrix systems for controlled release, cellulose ethers are uniformly incorporated throughout the tablet. Upon contact with water, the outer tablet skin is partially hydrated, forming a gel layer. The rate of diffusion of actives out of the gel layer and the rate of erosion determine the overall tablet dissolution and drug delivery rates. Precise and reliable adjustments of these rates are possible because the properties of cellulose ethers have been documented.

Cellulose ethers can be heated and mixed with plasticizers for extrusion or molding into a wide range of physical forms. Formulators use cellulose ether products to design single-unit matrix tablets, soft gel capsule replacements, and multi-particle delivery systems using extruded beads or shaped chips.

The preferred embodiments comprise a metal chlorophyllin chelate, which is a stable salt derivative of chlorophyll. Naturally occurring chlorophyll can exist as chlorophyll a, chlorophyll b, chlorophyll c, and chlorophyll d. Chlorophyll a and chlorophyll b are derived from plants and algae. A structure of chlorophyll is shown below.

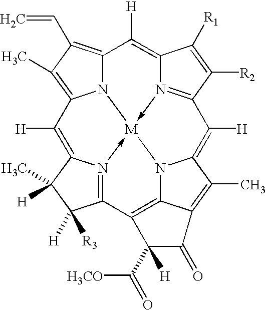

| | R₁ | R₂ | R₃ |
|---|---|---|---|
| chlorophyll a | CH₃ | CH₂CH₃ | X |
| chlorophyll b | CHO | CH₂CH₃ | X |

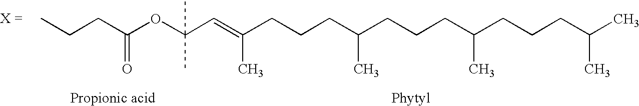

M = divalent metal

Chlorophyllin can be synthesized from chlorophyll by careful alkaline hydrolysis of chlorophyll. The hydrolysis opens the cyclopentanone ring and replaces the methyl and phytyl ester groups with a monovalent metal. In the preferred embodiments, metals used to form a chelate with chlorophyllin can be any metal that forms a stable complex with chlorophyllin. Preferably, the metals accommodate the divalent nature of the chlorophyllin structure. More preferably, the divalent metal is copper, cobalt, manganese, chromium, iron, nickel, zinc, or magnesium. Other metals can be present to bond with other functional groups present in the chlorophyllin structure. For example, any monovalent metal can bond with any carboxylic acid groups present in the chlorophyllin structure for solvation purposes. Preferably, metals to aid in solvation are lithium, sodium, magnesium, potassium, and calcium. In the preferred embodiments, the chlorophyllin can be derived from any form of chlorophyll, preferably chlorophyll a or chlorophyll b.

Chlorophyllin has shown a potent antimutagen capacity in different assays mainly achieved in vitro. E. Madrigal-Bujaidar et al. (Chlorophyllin inhibition of the genotoxic damage produced by benzo(a)pyrene in vivo, 17 Cancer Detect. Prev. 1249 (1993)) has shown that chlorophyllin can modulate the frequency of sister chromatid exchanges (SCE) damage induced by benzo(a)pyrene, giving about 80% of genotoxic inhibition. Chlorophyllin plus benzo(a)pyrene produced an inhibition of the cell proliferation kinetics (CPK). Furthermore, chlorophyllin is an inert compound to the body.

Additionally, chlorophyllin has a deodorizing effect and can mask certain smells of compounds in formulations to present more acceptable products to a consumer. In most cases, the objectionable smells from substances are barely detectable after being coated with chlorophyllin. Another benefit from coating a substance with chlorophyllin is the favorable appearance, usually a green color with resistance to fading.

In a certain embodiment, a solvent is added to help solubilize a saccharide polymer. Any solvent that can help solubilize the saccharide polymer can be used in the preferred embodiments. Preferable solvents include, but are not limited to, water, alcohols, ethyl acetate, glycerin, and mixtures thereof. Preferable alcohols include, but are not limited to, methanol, ethanol, propanol, isopropanol, butanol, isobutanol, tertiary butanol, and mixtures thereof. Additionally, additives can be included in the solution. Examples of additives include, but are not limited to, sugars, sweeteners, preservatives, colorants, flavorings, and buffers.

In a certain embodiment, after the saccharide polymer is suspended in an organic solvent, water, preferably deionized/filtered water, is added and mixed until the saccharide polymer dissolves, thereby forming a saccharide polymer solution. Preferably, the resulting solution has a pH of about 2 to 10, more preferably about 5 to 9. The resulting solution comprises about 1 to 10% saccharide polymer, about 60 to 90% organic solvent, and about 10 to 30% water; preferably, about 1 to 5% saccharide polymer, about 70 to 80% organic solvent, and about 15 to 25% water; and most preferably, about 4% saccharide polymer, about 76% organic solvent, and about 20% water. In preferred embodiments, the saccharide polymer is a cellulose ether and the organic solvent is an alcohol.

After the saccharide polymer is in solution, a metal chlorophyllin chelate is added and mixed into solution. In a certain embodiment, sodium copper chlorophyllin is added to the saccharide polymer solution while mixing. In an embodiment, sodium copper chlorophyllin is added at the concentration of about 5 to 500 grams of sodium copper chlorophyllin per about 1 gallon of saccharide polymer solution. Preferably, about 10 to 250 grams of sodium copper chlorophyllin is added to about 1 gallon of saccharide polymer solution. More preferably, about 40 to 70 grams of sodium copper chlorophyllin is added to about 1 gallon of saccharide polymer solution. The resulting solution is applied to a compound or substance, preferably in a spray dry application. Any method that can apply a coating of the solution and subsequently dry the coat can be used. Preferably, the spray dry application uses about 60° C. heated air moving at about 2400 cfm in a rotating stainless steel perforated pan. The solution is vaporized leaving a film containing a cellulose ether and an antimutagenic compound.

The disclosure below is of specific example setting forth a preferred method for making preferred compounds. This example is not intended to limit the scope, but rather to exemplify a certain embodiment.

### EXAMPLE 1

### Preparation of a Coating

A natural coating for use in coating compounds and substances is made by suspending METHOCEL™ E15 (Dow Chemical) in denatured alcohol while mixing. After the cellulose ether is suspended, water, preferably deionized/filtered water, is added and mixed until the cellulose ether dissolves. The resulting solution comprises about 4% METHOCEL™ E15, about 76% denatured alcohol, and about 20% water. After METHOCEL™ E15 is in solution, sodium copper chlorophyllin is added in an amount of about 54 grams per gallon of liquid while mixing. The resulting solution is applied in a spray dry application using about 60° C. heated air moving at about 2400 cfin in a rotating stainless steel perforated pan. The hydro alcohol content is flashed off, leaving a thin film containing the chlorophyllin.

Before the present natural coating, method making thereof, and method of use thereof are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

What is claimed is:

1. A coated substance comprising:
the substance; and
a coating surrounding the substance, said coating comprising a mixture of cellulose ether and a metal chlorophyllin chelate.

2. The coated substance of claim 1, wherein the cellulose ether is methylcellulose.

3. The coated substance of claim 1, wherein the cellulose ether is hydroxypropyl methylcellulose.

4. The coated substance of claim 1, wherein the metal chlorophyllin chelate is sodium copper chlorophyllin.

5. The coated substance of claim 1, wherein the substance is a pharmaceutical composition, dietary supplement, or nutraceutical.

6. A method for coating a substance to improve stability in the substance and to reduce objectionable tastes and/or odors in the substance comprising:
forming a solution of a cellulose ether;
adding a metal chlorophyllin chelate to the cellulose ether solution, thereby forming an chlorophyllin compound/cellulose ether solution;
applying the metal chlorophyllin chelate/cellulose ether solution to the substance; and
vaporizing the solvent from the metal chlorophyllin chelate/cellulose ether solution, thereby forming a coating on the substance.

7. The method of claim 6, wherein the cellulose ether is methylcellulose.

8. The method of claim 6, wherein the cellulose ether is hydroxypropyl methylcellulose.

9. The method of claim 6, wherein the metal chlorophyllin chelate is sodium copper chlorophyllin.

10. The method of claim 9, wherein sodium copper chlorophyllin is added to the cellulose ether solution at a concentration of about 5 to 500 grams of sodium copper chlorophyllin per about 1 gallon of cellulose ether solution.

11. The method of claim 10, wherein sodium copper chlorophyllin is added to the cellulose ether solution at a concentration of about 10 to 250 grams of sodium copper chlorophyllin per about 1 gallon of cellulose ether solution.

12. The method of claim 11, wherein sodium copper chlorophyllin is added to the cellulose ether solution at a concentration of about 40 to 70 grams of sodium copper chlorophyllin per about 1 gallon of cellulose ether solution.

13. The method of claim 6, wherein applying the metal chlorophyllin chelate/cellulose ether solution to the substance is performed by a spray dry application.
